(19) United States
(12) Patent Application Publication
(10) Pub. No.: US 2015/0112605 A1
(43) Pub. Date: Apr. 23, 2015

(54) SYSTEMS AND METHODS FOR GENERATING RESPIRATION ALARMS

(71) Applicant: Covidien LP, Mansfield, MA (US)

(72) Inventors: James N. Watson, Dunfermline (GB); Paul S. Addison, Edinburgh (GB)

(21) Appl. No.: 14/519,045

(22) Filed: Oct. 20, 2014

Related U.S. Application Data
(60) Provisional application No. 61/893,841, filed on Oct. 21, 2013.

(51) Int. Cl. G06F 19/00; G01N 33/00; A61B 5/08; A61B 5/1495; A61B 5/1455

(52) U.S. Cl. CPC G06F 19/3406; A61B 5/1495; A61B 5/14552; A61B 5/08; G01N 33/0036
USPC 702/19

(57) ABSTRACT

Systems and methods are provided for generating respiration alarms. Respiration information and oxygen saturation information is determined from a photoplethysmograph (PPG) signal. This information is analyzed in connection with activating a respiration lost alarm.

### CROSS REFERENCE TO RELATED APPLICATION

This application claims the benefit of U.S. Provisional Application No. 61/893,841, filed Oct. 21, 2013, which is hereby incorporated by reference herein in its entirety.

### SUMMARY

The present disclosure relates to physiological signal processing, and more particularly, the present disclosure relates to generating respiration alarms.

The present disclosure provides embodiments for a computer-implemented method comprising: receiving a photoplethysmograph (PPG) signal; generating, using processing circuitry, respiration information based on the PPG signal; generating oxygen saturation information based on the PPG signal; determining using processing circuitry, whether a respiration signal has been lost based on the oxygen saturation information and on the respiration information; and activating, using processing circuitry, a respiration lost alarm when it is determined that the respiration signal has been lost.

The present disclosure provides embodiments for a system comprising: an input for receiving a photoplethysmograph (PPG) signal; and processing circuitry configured for: generating respiration information based on the PPG signal, generating oxygen saturation information based on the PPG signal, determining whether a respiration signal has been lost based on the oxygen saturation information and on the respiration information, and activating a respiration lost alarm when it is determined that the respiration signal has been lost.

The present disclosure provides embodiments for a non-tangible computer-readable medium having computer program instructions stored thereon for performing the method comprising: receiving a photoplethysmograph (PPG) signal;

generating respiration information based on the PPG signal; generating oxygen saturation information based on the PPG signal; determining whether a respiration signal has been lost based on the oxygen saturation information and on the respiration information; and activating a respiration lost alarm when it is determined that the respiration signal has been lost.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows an illustrative patient monitoring system in accordance with some embodiments of the present disclosure;

FIG. 2 is a block diagram of the illustrative patient monitoring system of FIG. 1 coupled to a patient in accordance with some embodiments of the present disclosure;

FIG. 3 shows an illustrative PPG signal that is modulated by respiration in accordance with some embodiments of the present disclosure;

FIG. 4 shows a comparison of portions of the illustrative PPG signal of FIG. 3 in accordance with some embodiments of the present disclosure;

FIG. 5 shows illustrative steps for determining respiration information from a PPG signal in accordance with some embodiments of the present disclosure;

FIG. 6 shows an illustrative PPG signal, a first derivative of the PPG signal, and a second derivative of the PPG signal in accordance with some embodiments of the present disclosure; and

FIG. 7 shows illustrative steps for determining that a patient has lost respiration.

### DETAILED DESCRIPTION OF THE FIGURES

The present disclosure is directed towards generating a respiration lost alarm. Respiration lost refers to a sufficiently high loss in respiratory signal amplitude that creates a loss in confidence in calculated respiration information, such as respiration rate. The loss in signal amplitude may be with respect to a system noise floor. A respiration lost state or occurrence may be caused by any of a number of factors.

The respiration lost alarm may be based on respiration information derived from any suitable physiological signal, such as a photoplethysmograph (PPG) signal. For example, metrics from the PPG indicative of respiration may be derived and analyzed to calculate respiration information such as respiration rate. These and other metrics may also be used to determine signal quality measures as an indication of a confidence of the respiration information.

A derived respiration information signal, such as a signal from which respiration rate may be readily derivable, may be susceptible to noise and may be particularly difficult to identify due to its low amplitude. The signal may therefore be effectively lost even when a subject is breathing very shallowly. Attaching a respiration lost alarm to this observation may therefore product frequent false alarms. However, a loss of a respiration information signal with an associated decrease in oxygen saturation may indicate the occurrence or the onset of a significant respiratory problem for the patient. In this instance, an alarm may be triggered to alert the clinician with greater specificity than an alarm based only on the respiration information signal. Such an alarm would also react faster to respiratory compromise than when based only on oxygen saturation.

For purposes of clarity, the present disclosure is written in the context of the physiological signal being a PPG signal generated by a pulse oximetry system. It will be understood that any other suitable physiological signal or any other suitable system may be used in accordance with the teachings of the present disclosure.

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient). Pulse oximeters may be included in patient monitoring systems that measure and display various blood flow characteristics including, but not limited to, the oxygen saturation of hemoglobin in arterial blood. Such patient monitoring systems may also measure and display additional physiological parameters, such as a patient's pulse rate.

An oximeter may include a light sensor that is placed at a site on a patient, typically a fingertip, toe, forehead or earlobe, or in the case of a neonate, across a foot. The oximeter may use a light source to pass light through blood perfused tissue and photoelectrically sense the absorption of the light in the tissue. In addition, locations that are not typically understood to be optimal for pulse oximetry serve as suitable sensor locations for the monitoring processes described herein, including any location on the body that has a strong pulsatile arterial flow. For example, additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, and around or in front of the ear. Suitable sensors for these locations may include sensors for sensing absorbed light based on detecting reflected light. In all suitable locations, for example, the oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some applications, the light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared (IR) wavelengths may be used because it has been observed that highly oxygenated blood will absorb relatively less Red light and more IR light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based at least in part on Lambert-Beer's law. The following notation will be used herein:

I(λ,t)=I₀(λ)exp(−(sβ₀(λ)+(1−s)βᵣ(λ))l(t)) (1)

where:
λ=wavelength;
t=time;
I=intensity of light detected;
I₀=intensity of light transmitted;
S=oxygen saturation;
β₀, β=empirically derived absorption coefficients; and
l(t)=a combination of concentration and path length from emitter to detector as a function of time.

The traditional approach measures light absorption at two wavelengths (e.g., Red and IR), and then calculates saturation by solving for the “ratio of ratios” as follows.

1. The natural logarithm of Eq. 1 is taken (“log” will be used to represent the natural logarithm) for IR and Red to yield

log I=log I₀−(sβ₀+(1−s)βᵣ)l. (2)

2. Eq. 2 is then differentiated with respect to time to yield

$$\frac{d log I}{d t} = - ( s {β}_{o} + ( 1 - s ) {β}_{r} ) \frac{d l}{d t} . \qquad ( 3 )$$

3. Eq. 3, evaluated at the Red wavelength λ_{R}, is divided by Eq. 3 evaluated at the IR wavelength λ_{IR} in accordance with

$$\frac{d log I ( {λ}_{R} ) / d t}{d log I ( {λ}_{IR} ) / d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} . \qquad ( 4 )$$

4. Solving for S yields

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{o} ( {λ}_{IR} ) - {β}_{r} ( {λ}_{IR} ) ) - \\ \frac{d log I ( {λ}_{IR} )}{d t} ( {β}_{o} ( {λ}_{R} ) - {β}_{r} ( {λ}_{R} ) )\end{matrix}} . \qquad ( 5 )$$

5. Note that, in discrete time, the following approximation can be made:

$$\frac{d log I ( λ , t )}{d t} ≃ log I ( λ , {t}_{2} ) - log I ( λ , {t}_{1} ) . \qquad ( 6 )$$

6. Rewriting Eq. 6 by observing that log A−log B=log(A/B) yields

$$\frac{d log I ( λ , t )}{d t} ≃ log ( \frac{I ( {t}_{2} , λ )}{I ( {t}_{1} , λ )} ) . \qquad ( 7 )$$

7. Thus, Eq. 4 can be expressed as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R , \qquad ( 8 )$$

where R represents the “ratio of ratios.”
8. Solving Eq. 4 for S using the relationship of Eq. 5 yields

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} . \qquad ( 9 )$$

9. From Eq. 8, R can be calculated using two points (e.g., PPG maximum and minimum), or a family of points. One method applies a family of points to a modified version of Eq. 8. Using the relationship

$$\frac{d log I}{d t} = \frac{d I / d t}{I} , \qquad ( 10 )$$

Eq. 8 becomes

$$\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{R} )}} = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} = R , \qquad ( 11 )$$

which defines a cluster of points whose slope of y versus X will give R when

x=[I(t₂,λ_{IR})−I(t₁,λ_{IR})]I(t₁,λ_{R}), (12)

and

y=[I(t₂,λ_{R})−I(t₁,λ_{R})]I(t₁,λ_{IR}). (13)

Once R is determined or estimated, for example, using the techniques described above, the blood oxygen saturation can be determined or estimated using any suitable technique for relating a blood oxygen saturation value to R. For example, blood oxygen saturation can be determined from empirical data that may be indexed by values of R, and/or it may be determined from curve fitting and/or other interpolative techniques.

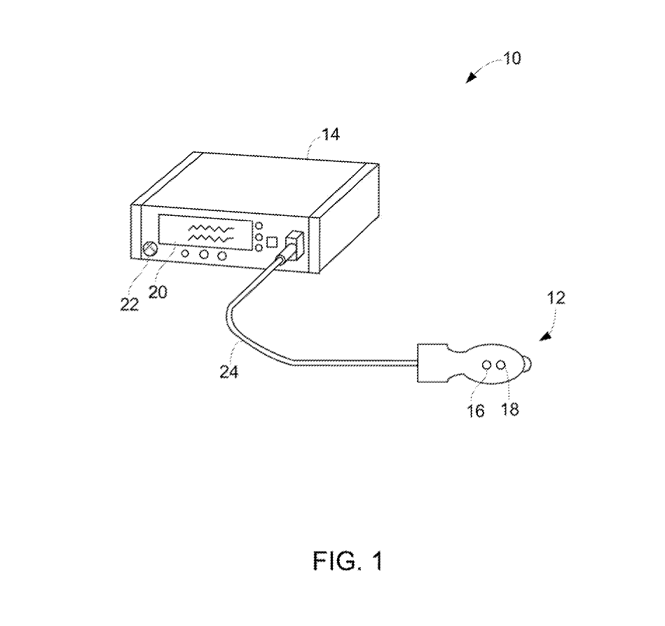

FIG. 1 is a perspective view of an embodiment of a patient monitoring system 10. System 10 may include sensor unit 12 and monitor 14. In some embodiments, sensor unit 12 may be part of an oximeter. Sensor unit 12 may include an emitter 16 for emitting light at one or more wavelengths into a patient's tissue. A detector 18 may also be provided in sensor unit 12 for detecting the light originally from emitter 16 that emanates from the patient's tissue after passing through the tissue. Any suitable physical configuration of emitter 16 and detector 18 may be used. In an embodiment, sensor unit 12 may include multiple emitters and/or detectors, which may be spaced apart. System 10 may also include one or more additional sensor units (not shown) that may take the form of any of the embodiments described herein with reference to sensor unit 12. An additional sensor unit may be the same type of sensor unit as sensor unit 12, or a different sensor unit type than sensor unit 12. Multiple sensor units may be capable of being positioned at two different locations on a subject's body; for example, a first sensor unit may be positioned on a patient's forehead, while a second sensor unit may be positioned at a patient's fingertip.

Sensor units may each detect any signal that carries information about a patient's physiological state, such as an electrocardiograph signal, arterial line measurements, or the pulsatile force exerted on the walls of an artery using, for example, oscillometric methods with a piezoelectric transducer. According to some embodiments, system 10 may include two or more sensors forming a sensor array in lieu of either or both of the sensor units. Each of the sensors of a sensor array may be a complementary metal oxide semiconductor (CMOS) sensor. Alternatively, each sensor of an array may be charged coupled device (CCD) sensor. In some embodiments, a sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier. It will be understood that any type of sensor, including any type of physiological sensor, may be used in one or more sensor units in accordance with the systems and techniques disclosed herein. It is understood that any number of sensors measuring any number of physiological signals may be used to determine physiological information in accordance with the techniques described herein.

In some embodiments, emitter 16 and detector 18 may be on opposite sides of a digit such as a finger or toe, in which case the light that is emanating from the tissue has passed completely through the digit. In some embodiments, emitter 16 and detector 18 may be arranged so that light from emitter 16 penetrates the tissue and is reflected by the tissue into detector 18, such as in a sensor designed to obtain pulse oximetry data from a patient's forehead.

In some embodiments, sensor unit 12 may be connected to and draw its power from monitor 14 as shown. In another embodiment, the sensor may be wirelessly connected to monitor 14 and include its own battery or similar power supply (not shown). Monitor 14 may be configured to calculate physiological parameters (e.g., pulse rate, blood oxygen saturation (e.g., SpO₂), and respiration information) based at least in part on data relating to light emission and detection received from one or more sensor units such as sensor unit 12 and an additional sensor (not shown). In some embodiments, the calculations may be performed on the sensor units or an intermediate device and the result of the calculations may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range. In some embodiments, the system 10 includes a stand-alone monitor in communication with the monitor 14 via a cable or a wireless network link.

In some embodiments, sensor unit 12 may be communicatively coupled to monitor 14 via a cable 24. In some embodiments, a wireless transmission device (not shown) or the like may be used instead of or in addition to cable 24. Monitor 14 may include a sensor interface configured to receive physiological signals from sensor unit 12, provide signals and power to sensor unit 12, or otherwise communicate with sensor unit 12. The sensor interface may include any suitable hardware, software, or both, which may allow communication between monitor 14 and sensor unit 12.

As is described herein, monitor 14 may generate a PPG signal based on the signal received from sensor unit 12. The PPG signal may consist of data points that represent a pulsatile waveform. The pulsatile waveform may be modulated based on the respiration of a patient. Respiratory modulations may include baseline modulations, amplitude modulations, frequency modulations, respiratory sinus arrhythmia, any other suitable modulations, or any combination thereof. Respiratory modulations may exhibit different phases, amplitudes, or both, within a PPG signal and may contribute to complex behavior (e.g., changes) of the PPG signal. For example, the amplitude of the pulsatile waveform may be modulated based on respiration (amplitude modulation), the frequency of the pulsatile waveform may be modulated based on respiration (frequency modulation), and a signal baseline for the pulsatile waveform may be modulated based on respiration (baseline modulation). Monitor 14 may analyze the PPG signal (e.g., by generating respiration morphology signals from the PPG signal, generating a combined autocorrelation sequence based on the respiration morphology signals, and calculating respiration information from the combined autocorrelation sequence) to determine respiration information based on one or more of these modulations of the PPG signal.

As is described herein, respiration information may be determined from the PPG signal by monitor 14. However, it will be understood that the PPG signal could be transmitted to any suitable device for the determination of respiration information, such as a local computer, a remote computer, a nurse station, mobile devices, tablet computers, or any other device capable of sending and receiving data and performing processing operations. Information may be transmitted from monitor 14 in any suitable manner, including wireless (e.g., WiFi, Bluetooth, etc.), wired (e.g., USB, Ethernet, etc.), or application-specific connections. The receiving device may determine respiration information as described herein.

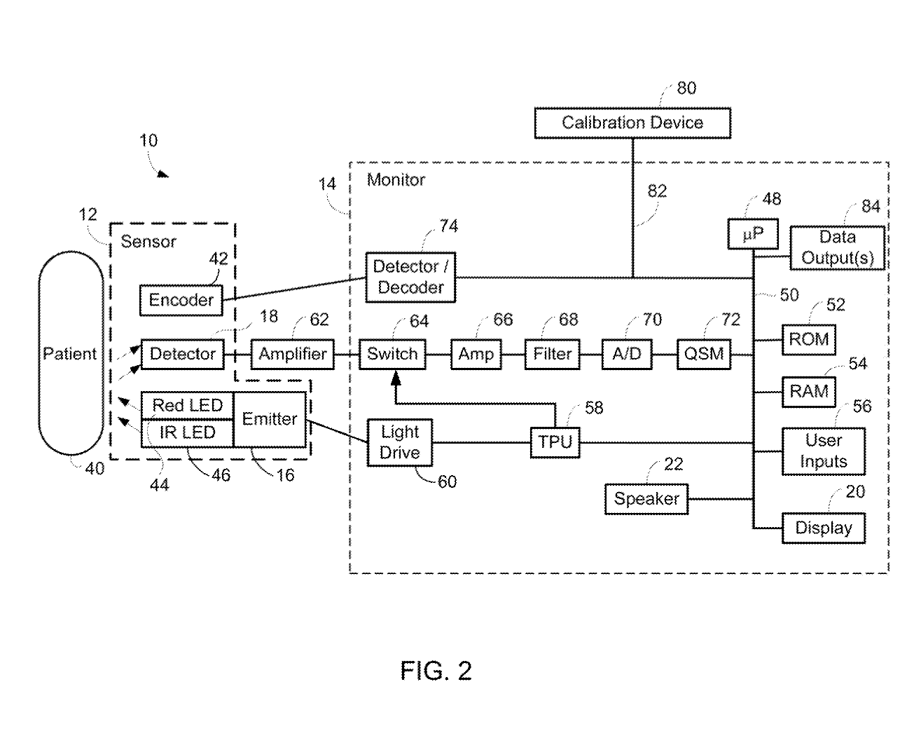

FIG. 2 is a block diagram of a patient monitoring system, such as patient monitoring system 10 of FIG. 1, which may be coupled to a patient 40 in accordance with an embodiment. Certain illustrative components of sensor unit 12 and monitor 14 are illustrated in FIG. 2.

Sensor unit 12 may include emitter 16, detector 18, and encoder 42. In the embodiment shown, emitter 16 may be configured to emit at least two wavelengths of light (e.g., Red and IR) into a patient's tissue 40. Hence, emitter 16 may include a Red light emitting light source such as Red light emitting diode (LED) 44 and an IR light emitting light source such as IR LED 46 for emitting light into the patient's tissue 40 at the wavelengths used to calculate the patient's physiological parameters. In some embodiments, the Red wavelength may be between about 600 nm and about 700 nm, and the IR wavelength may be between about 800 nm and about 1000 nm. In embodiments where a sensor array is used in place of a single sensor, each sensor may be configured to emit a single wavelength. For example, a first sensor may emit only a Red light while a second sensor may emit only an IR light. In a further example, the wavelengths of light used may be selected based on the specific location of the sensor.

It will be understood that, as used herein, the term “light” may refer to energy produced by radiation sources and may include one or more of radio, microwave, millimeter wave, infrared, visible, ultraviolet, gamma ray or X-ray electromagnetic radiation. As used herein, light may also include electromagnetic radiation having any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In some embodiments, detector 18 may be configured to detect the intensity of light at the Red and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the Red and IR wavelengths in the patient's tissue 40.

In some embodiments, encoder 42 may contain information about sensor unit 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information about a patient's characteristics may allow monitor 14 to determine, for example, patient-specific threshold ranges in which the patient's physiological parameter measurements should fall and to enable or disable additional physiological parameter algorithms. This information may also be used to select and provide coefficients for equations from which measurements may be determined based at least in part on the signal or signals received at sensor unit 12. For example, some pulse oximetry sensors rely on equations to relate an area under a portion of a PPG signal corresponding to a physiological pulse to determine blood pressure. These equations may contain coefficients that depend upon a patient's physiological characteristics as stored in encoder 42.

Encoder 42 may, for instance, be a coded resistor that stores values corresponding to the type of sensor unit 12 or the type of each sensor in the sensor array, the wavelengths of light emitted by emitter 16 on each sensor of the sensor array, and/or the patient's characteristics and treatment information. In some embodiments, encoder 42 may include a memory on which one or more of the following information may be stored for communication to monitor 14; the type of the sensor unit 12; the wavelengths of light emitted by emitter 16; the particular wavelength each sensor in the sensor array is monitoring; a signal threshold for each sensor in the sensor array; any other suitable information; physiological characteristics (e.g., gender, age, weight); or any combination thereof.

In some embodiments, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, data output 84, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium that can be used to store the desired information and that can be accessed by components of the system.

In the embodiment shown, a time processing unit (TPU) 58 may provide timing control signals to light drive circuitry 60, which may control when emitter 16 is illuminated and multiplexed timing for Red LED 44 and IR LED 46. TPU 58 may also control the gating-in of signals from detector 18 through amplifier 62 and switching circuit 64. These signals are sampled at the proper time, depending upon which light source is illuminated. The received signal from detector 18 may be passed through amplifier 66, low pass filter 68, and analog-to-digital converter 70. The digital data may then be stored in a queued serial module (QSM) 72 (or buffer) for later downloading to RAM 54 as QSM 72 is filled. In some embodiments, there may be multiple separate parallel paths having components equivalent to amplifier 66, filter 68, and/or A/D converter 70 for multiple light wavelengths or spectra received. Any suitable combination of components (e.g., microprocessor 48, RAM 54, analog to digital converter 70, any other suitable component shown or not shown in FIG. 2) coupled by bus 50 or otherwise coupled (e.g., via an external bus), may be referred to as “processing equipment” or “processing circuitry”.

In some embodiments, microprocessor 48 may determine the patient's physiological parameters, such as SpO₂, pulse rate, and/or respiration information, using various algorithms and/or look-up tables based on the value of the received signals and/or data corresponding to the light received by detector 18. As is described herein, microprocessor 48 may generate respiration morphology signals and determine respiration information from a PPG signal.

Signals corresponding to information about patient 40, and particularly about the intensity of light emanating from a patient's tissue over time, may be transmitted from encoder 42 to decoder 74. These signals may include, for example, encoded information relating to patient characteristics. Decoder 74 may translate these signals to enable microprocessor 48 to determine the thresholds based at least in part on algorithms or look-up tables stored in ROM 52. In some embodiments, user inputs 56 may be used to enter information, select one or more options, provide a response, input settings, any other suitable inputting function, or any combination thereof. User inputs 56 may be used to enter information about the patient, such as age, weight, height, diagnosis, medications, treatments, and so forth. In some embodiments, display 20 may exhibit a list of values, which may generally apply to the patient, such as, for example, age ranges or medication families, which the user may select using user inputs 56.

Calibration device 80, which may be powered by monitor 14 via a communicative coupling 82, a battery, or by a conventional power source such as a wall outlet, may include any suitable signal calibration device. Calibration device 80 may be communicatively coupled to monitor 14 via communicative coupling 82, and/or may communicate wirelessly (not shown). In some embodiments, calibration device 80 is completely integrated within monitor 14. In some embodiments, calibration device 80 may include a manual input device (not shown) used by an operator to manually input reference signal measurements obtained from some other source (e.g., an external invasive or non-invasive physiological measurement system).

Data output 84 may provide for communications with other devices utilizing any suitable transmission medium, including wireless (e.g., WiFi, Bluetooth, etc.), wired (e.g., USB, Ethernet, etc.), or application-specific connections. Data output 84 may receive messages to be transmitted from microprocessor 48 via bus 50. Exemplary messages to be sent in an embodiment described herein may include samples of the PPG signal to be transmitted to an external device for determining respiration information.

The optical signal attenuated by the tissue of patient 40 can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. Also, because blood is a fluid, it responds differently than the surrounding tissue to inertial effects, which may result in momentary changes in volume at the point to which the oximeter probe is attached.

Noise (e.g., from patient movement) can degrade a sensor signal relied upon by a care provider, without the care provider's awareness. This is especially true if the monitoring of the patient is remote, the motion is too small to be observed, or the care provider is watching the instrument or other parts of the patient, and not the sensor site. Processing sensor signals (e.g., PPG signals) may involve operations that reduce the amount of noise present in the signals, control the amount of noise present in the signal, or otherwise identify noise components in order to prevent them from affecting measurements of physiological parameters derived from the sensor signals.

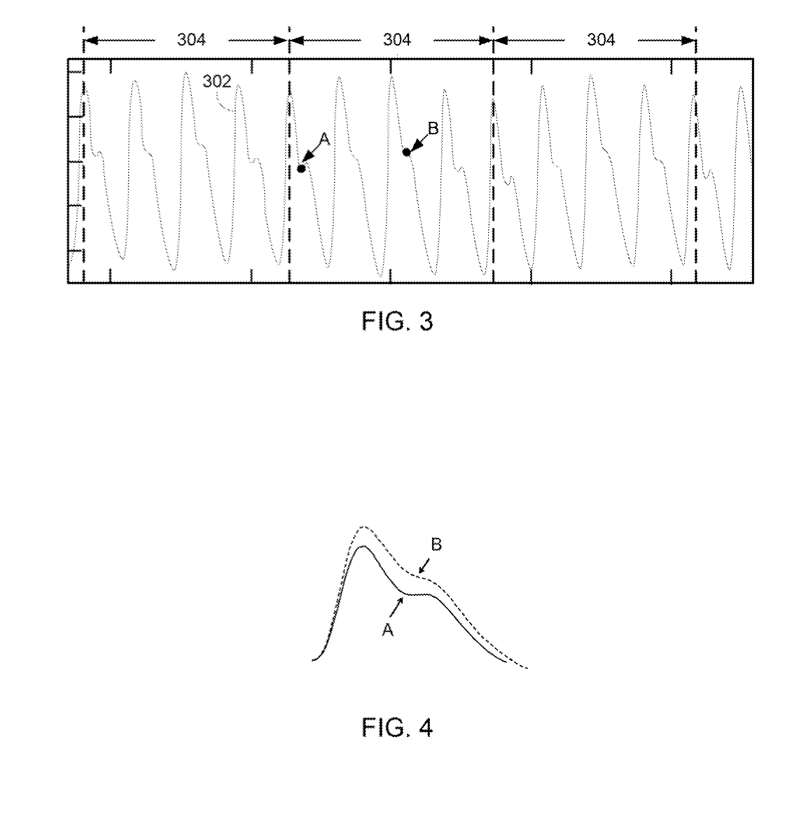

FIG. 3 shows an illustrative PPG signal 302 that is modulated by respiration in accordance with some embodiments of the present disclosure. PPG signal 302 may be a periodic signal that is indicative of changes in pulsatile blood flow. Each cycle of PPG signal 302 may generally correspond to a pulse, such that a heart rate may be determined based on PPG signal 302. Each respiratory cycle 304 may correspond to a breath. The period of a respiratory cycle may typically be longer than the period of a pulsatile cycle, such that any changes in the pulsatile blood flow due to respiration occur over a number of pulsatile cycles. The volume of the pulsatile blood flow may also vary in a periodic manner based on respiration, resulting in modulations to the pulsatile blood flow such as amplitude modulation, frequency modulation, and baseline modulation. This modulation of PPG signal 302 due to respiration may result in changes to the morphology of PPG signal 302.

FIG. 4 shows a comparison of portions of the illustrative PPG signal 302 of FIG. 3 in accordance with some embodiments of the present disclosure. The signal portions compared in FIG. 4 may demonstrate differing morphology due to respiration modulation based on the relative location of the signal portions within a respiratory cycle 304. For example, a first pulse associated with the respiratory cycle may have a relatively low amplitude (indicative of amplitude and baseline modulation) as well as an obvious distinct dichrotic notch as indicated by point A. A second pulse may have a relatively high amplitude (indicative of amplitude and baseline modulation) as well as a dichrotic notch that has been washed out as depicted by point B. Frequency modulation may be evident based on the relative period of the first pulse and second pulse. Referring again to FIG. 3, by the end of the respiratory cycle 304 the pulse features may again be similar to the morphology of A. Although the impact of respiration modulation on the morphology of a particular PPG signal 302 has been described herein, it will be understood that respiration may have varied effects on the morphology of a PPG signal other than those depicted in FIGS. 3 and 4.

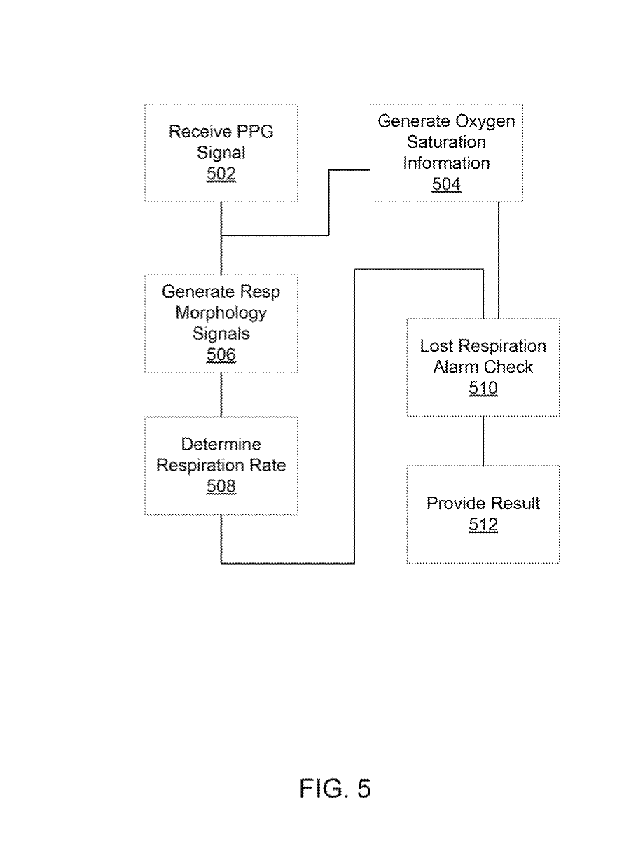

FIG. 5 shows illustrative steps for determining respiration information from a PPG signal in accordance with some embodiments of the present disclosure. Although exemplary steps are described herein, it will be understood that steps may be omitted and that any suitable additional steps may be added for determining respiration information. Although the steps described herein may be performed by any suitable device, in an exemplary embodiment, the steps may be performed by monitoring system 10. At step 502, monitoring system 10 may receive a PPG signal as described herein. Although the PPG signal may be processed in any suitable manner, in an embodiment, the PPG signal may be analyzed each 5 seconds, and for each 5 second analysis window, the most recent 45 seconds of the PPG signal may be analyzed.

At step 504, monitoring system 10 may determine oxygen saturation from the PPG signal. Although it will be understood that oxygen saturation may be determined from a PPG signal in any suitable manner, in an embodiment a desired signal portion or portions of the PPG signal (e.g., corresponding to arterial and/or venous blood flow) may be separated from an undesired signal portion or portions (e.g., corresponding to patient movement and/or system noise) and oxygen saturation may be determined as described herein (e.g., based on the ratio of ratios) from the desired signal portion or portions. In some embodiments, the oxygen saturation values may be stored in memory. Although stored oxygen saturation values may be utilized in any suitable manner (e.g., to generate data trend information for transmission or display), in an embodiment, the stored oxygen saturation values may be used as a factor to determine whether to initiate an alarm based on lost respiration as described herein.

At step 506, monitoring system 10 may generate one or more respiration morphology signals from the PPG signal. In some embodiments, a plurality of respiration morphology signals may be generated from the PPG signal, such as a down respiration morphology signal, a delta of second derivative (DSD) respiration morphology signal, and a kurtosis respiration morphology signal. Although a respiration morphology signal may be generated in any suitable manner, in an embodiment, each respiration morphology signal may be generated based on calculating a series of morphology metrics from a PPG signal. One or more morphology metrics maybe calculated for each portion of the PPG signal (e.g., for each fiducial defined portion), a series of morphology metrics may be calculated over time, and the series of morphology metrics may be processed to generate one or more morphology metric signals.

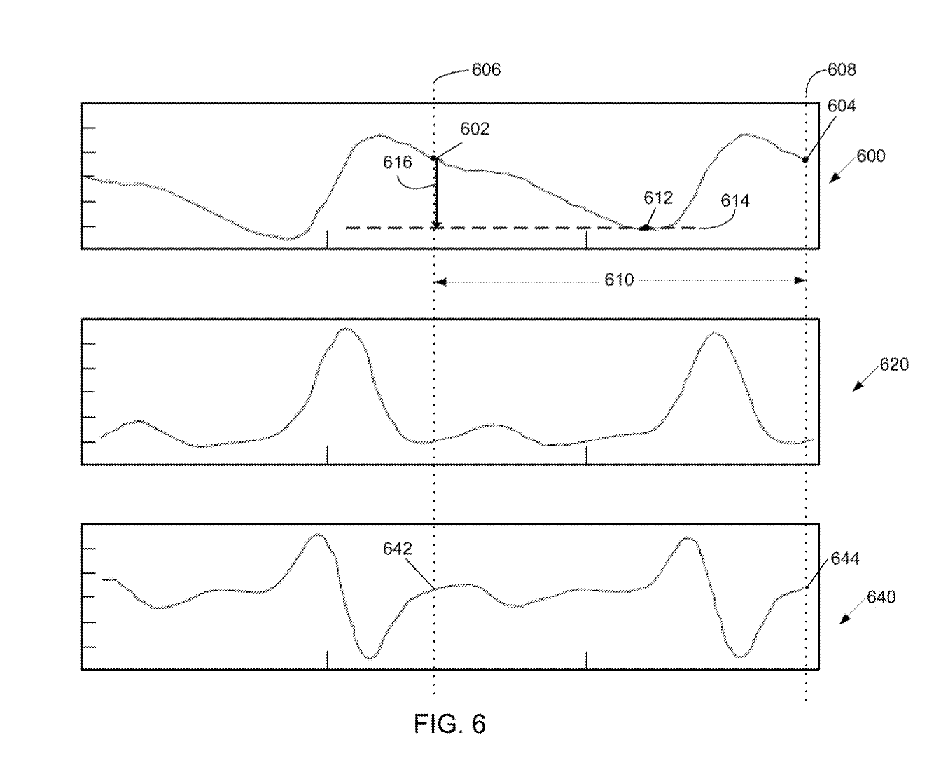

FIG. 6 depicts exemplary signals used for calculating morphology metrics from a received PPG signal. The abscissa of each plot of FIG. 6 may represent time and the ordinate of each plot may represent magnitude. PPG signal 600 may be a received PPG signal, first derivative signal 620 may be a signal representing the first derivative of the PPG signal 600, and second derivative signal 640 may be a signal representing the second derivative of the PPG signal 600. As will be described herein, morphology metrics may be calculated for portions of these signals, and a series of morphology metrics calculated over time may be processed to generate the respiration morphology signals. Although particular morphology metric calculations are set forth below, each of the morphology metric calculations may be modified in any suitable manner.

Although morphology metrics may be calculated based on any suitable portions of the PPG signal 600 (as well as the first derivative signal 620, second derivative signal 640, and any other suitable signals that may be generated from the PPG signal 600), in an exemplary embodiment, morphology metrics may be calculated for each fiducial-defined portion such as fiducial defined portion 610 of the PPG signal 600. Exemplary fiducial points 602 and 604 are depicted for PPG signal 600, and fiducial lines 606 and 608 demonstrate the location of fiducial points 602 and 604 relative to first derivative signal 620 and second derivative signal 640.

Although it will be understood that fiducial points may be identified in any suitable manner, in exemplary embodiments fiducial points may be identified based on features of the PPG signal 620 or any derivatives thereof (e.g., first derivative signal 620 and second derivative signal 640) such as peaks, troughs, points of maximum slope, dichrotic notch locations, pre-determined offsets, any other suitable features, or any combination thereof. Fiducial points 602 and 604 may define a fiducial-defined portion 610 of PPG signal 600. The fiducial points 602 and 604 may define starting and ending points for determining morphology metrics, and the fiducial-defined portion 610 may define a relevant portion of data for determining morphology metrics. It will be understood that other starting points, ending points, and relative portions of data may be utilized to determine morphology metrics.

An exemplary morphology metric may be a down metric. The down metric is the difference between a first (e.g., fiducial) sample of a fiducial-defined portion (e.g., fiducial defined portion 610) of the PPG signal (e.g., PPG signal 600) and a minimum sample (e.g., minimum sample 612) of the fiducial-defined portion 610 of the PPG signal 600. The down metric may also be calculated based on other points of a fiducial-defined portion. The down metric is indicative of physiological characteristics which are related to respiration, e.g., amplitude and baseline modulations of the PPG signal. In an exemplary embodiment, fiducial point 602 defines the first location for calculation of a down metric for fiducial-defined portion 610. In the exemplary embodiment, the minimum sample of fiducial-defined portion 610 is minimum point 612, and is indicated by horizontal line 614. The down metric may be calculated by subtracting the value of minimum point 612 from the value of fiducial point 602, and is depicted as down metric 616.

Another exemplary morphology metric may be a kurtosis metric for a fiducial-defined portion. Kurtosis measures the peakedness of the PPG signal 600 or a derivative thereof (e.g., first derivative signal 620 or second derivative signal 640). In an exemplary embodiment, the kurtosis metric may be based on the peakedness of the first derivative signal 620. The peakedness is sensitive to both amplitude and period (frequency) changes, and may be utilized as an input to generate respiration morphology signals that may be used to determine respiration information such as respiration rate. Kurtosis may be calculated based on the following formulae:

$$D = \frac{1}{n} {\sum }_{i = 1}^{n} {( {x}_{i}^{′} - \overline{{x}^{′}} )}^{2}$$

$$Kurtosis = \frac{1}{{nD}^{2}} {\sum }_{i = 1}^{n} {( {x}_{i}^{′} - \overline{{x}^{′}} )}^{4}$$

where:
xᵢ′=ith sample of 1^{st} derivative;
x̅′̅=mean of 1st derivative of fiducial-defined portion;
n=set of all samples in the fiducial-defined portion

Another exemplary morphology metric may be a delta of the second derivative (DSD) between consecutive fiducial-defined portions, e.g., at consecutive fiducial points. Measurement points 642 and 644 for a DSD calculation are depicted at fiducial points 602 and 604 as indicated by fiducial lines 606 and 608. The second derivative signal is indicative of the curvature of a signal. Changes in the curvature of the PPG signal 600 that can be identified with second derivative signal 640 are indicative of changes in internal pressure that occur during respiration, particularly changes near the peak of a pulse. By providing a metric of changes in curvature of the PPG signal, the DSD morphology metric may be utilized as an input to determine respiration information, such as respiration rate. The DSD metric may be calculated for each fiducial-defined portion by identifying the value of the second derivative signal 640 at the current fiducial point (e.g., fiducial point 642 of fiducial-defined portion 610) and subtracting from that the value of the second derivative signal 640 at the next fiducial point (e.g., fiducial point 644 of fiducial-defined portion 610).

Although a down metric, kurtosis metric, and DSD metric have been described, any suitable morphology metrics related to respiration may be calculated for use in generating respiration morphology signals. Other exemplary morphology metrics that may be relevant to determining a physiological parameter such as respiration information from a PPG signal may include an up metric, a skew metric, a ratio of samples metric (e.g., a b/a ratio metric or c/a ratio metric), a i_b metric, a peak amplitude metric, a center of gravity metric, and an area metric. It will be understood that metrics may be determined from the original PPG signal or any derivative thereof (e.g., a down metric may be determined for each of the PPG signal, the first derivative of the PPG signal, and/or the second derivative of the PPG signal).

In some embodiments, each series of morphology metric values may be further processed in any suitable manner to generate the respiration morphology signals. Although any suitable processing operations may be performed for each series of morphology metric values, in an exemplary embodiment, each series of morphology metric values may be filtered (e.g., based on frequencies associated with respiration) and interpolated to generate the plurality of respiration morphology signals. Processing may then continue to step 508.

At step 508, monitoring system may calculate respiration information such as respiration rate. Although respiration rate may be calculated in any suitable manner, in some embodiments, respiration rate may be calculated based on the down respiration morphology signal, the kurtosis respiration morphology signal, and the DSD respiration morphology signal. In an embodiment, an autocorrelation sequence may be generated for each of the respiration morphology signals. The peaks of an autocorrelation correspond to portions of the signal that include the same or similar information. Thus, the peaks of the autocorrelation sequences may correspond to periodic aspects of the underlying respiration morphology signals, which in turn may correspond to respiration information such as respiration rate.

Although it will be understood that respiration information such as respiration rate may be determined from one or more of the autocorrelation sequences in any suitable manner, in an embodiment, the autocorrelation sequences may be combined to generate a combined autocorrelation sequence and the respiration rate may be determined based on a lag (i.e., time delay associated with the period of breathing) associated with a peak of the autocorrelation sequence. Although the autocorrelation sequences may be combined in any suitable manner, in an exemplary embodiment the autocorrelation sequences having the most periodic information may be given the greatest weight in the combination.

Although it will be understood that a peak of the combined autocorrelation sequence may be selected as corresponding to respiration rate in any suitable manner, in an embodiment, the maxima of the peaks may be compared to a threshold, and if one or more peaks exceed the threshold, the first of the peaks that exceeds the threshold may be selected. Respiration rate may then be determined from the selected peak based on the lag associated with the peak. In some embodiments, the respiration rate values may be stored in memory. Although stored respiration values may be utilized in any suitable manner (e.g., to generate data trend information for transmission or display), in an embodiment, the stored oxygen saturation values may be used as a factor to determine whether to initiate an alarm based on lost respiration as described herein. Processing may then continue to step 510.

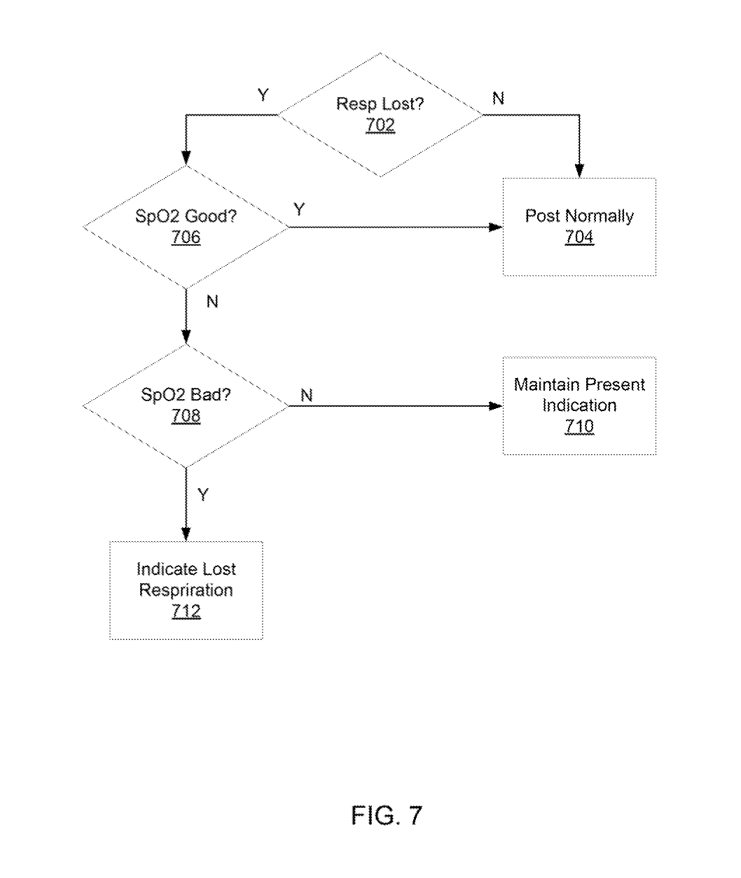

At step 510, monitor 10 may check a lost respiration alarm status. Although the lost respiration alarm status may be checked in any suitable manner, in an embodiment, the alarm status may be based on the determination of respiration rate and oxygen saturation as described in FIG. 7.

FIG. 7 depicts illustrative steps for determining a lost respiration alarm status in accordance with some embodiments of the present disclosure. Although exemplary steps are described herein, it will be understood that steps may be omitted and that any suitable additional steps may be added for determining a lost respiration alarm status. Although the steps described herein may be performed by any suitable device, in an exemplary embodiment, the steps may be performed by monitoring system 10.

At step 702, monitoring system 10 may analyze determined respiration information to determine whether the respiration has been lost. Although lost respiration may be identified in any suitable manner, in some embodiments, it may be determined that respiration rate cannot be determined from a received signal or that a respiration rate determined from a received signal is associated with a low confidence level that the respiration rate is correct (e.g., a confidence level associated with a respiration rate measurement is less than a threshold confidence). It will be understood that either one or both of these situations is being referred to herein when the term “respiration lost”, “lost respiration”, or any such similar term is used in this disclosure.

Although it may be determined that respiration rate cannot be determined from a received signal in any suitable manner, in some embodiments, the determination may be based on analysis of the respiration morphology signals, autocorrelation sequences, combined autocorrelation sequence, any other signal or analysis used to determine respiration rate, historical respiration rate information, or any combination thereof. For example, respiration may be considered lost when a signal representative of respiration information, such as respiration rate, has relatively low amplitude when compared to, for example, a system noise floor. In some embodiments, lost respiration may be indicated when no peak of the combined autocorrelation sequence exceeds a threshold. In some embodiments, lost respiration may be indicated based on no peak of the combined autocorrelation sequence exceeding a threshold for at least a predetermined elapsed time.

Although it may be determined that a respiration rate determined from a received signal is associated with a low confidence level in any suitable manner, in some embodiments, the determination may be based on analysis of the respiration morphology signals, autocorrelation sequences, combined autocorrelation sequence, any other signal or analysis used to determine respiration rate, historical respiration rate information, or any combination thereof. In some embodiments, a confidence value may be determined for each of the respiration morphology signals and/or autocorrelation sequences. If none of the confidence values meet a minimum threshold, or if a combined confidence value does not meet a minimum threshold, lost respiration may be indicated. In some embodiments, a confidence value may be based on a pattern of historical respiration rate values. For example, a confidence value may be based on the variability of respiration rate values over time (e.g., the standard deviation of a set of recent respiration rate values), where a high variability (low confidence) indicates a possibility that the respiration rate values are not based on actual respiration. In some embodiments, confidence values may be adjustable based on user inputs, patient history, historical values, other patient information, any other suitable information, or any combination thereof.

In some embodiments, the elapsed time that respiration is determined to be lost may also be a factor in determining a confidence of computed respiration information and in triggering a respiration lost alarm. For example, confidence in respiration information may be determined based on an analysis of the duration that respiration has been lost, the degree to which a respiration signal amplitude being relied on in determining whether respiration is lost (e.g., the combined autocorrelation signal discussed above) is below a threshold, or both. In this example, an integral may be calculated representative of the area between a respiration signal amplitude measurement over time and a corresponding threshold. When the integral exceeds a threshold, an alarm indicating that the respiration is lost may be triggered.

If it is determined that respiration was not lost at step 702, processing may continue to step 704. At step 704, monitor 10 may set a lost respiration alarm status to post normally. Although a lost respiration alarm status may be set to post normally in any suitable manner, in an embodiment, any alarms (e.g., visual alarms, audio alarms, and alarm messages) relating to lost respiration may be reset.

If it is determined that respiration was lost at step 702, processing may continue to step 706. As described herein, steps 706 and 708 may utilize determined oxygen saturation values to further analyze lost respiration.

At step 706, oxygen saturation values may be analyzed to determine whether, in spite of the indication of lost respiration, any lost respiration alarm should be suppressed or switched off. For example, if oxygen saturation is increasing it may be assumed that the patient is receiving adequate oxygen. In some embodiments, if the recently determined oxygen saturation value exceeds the previous value by more than a threshold, a lost respiration alarm may be suppressed or switched off. In some embodiments, a trend of recently determined oxygen saturation values may be analyzed. Although the trend of recently determined oxygen saturation values may be analyzed in any suitable manner, in an embodiment, an average oxygen saturation value (e.g., for 5 second segments of oxygen saturation) may be calculated for a snapshot of recently determined oxygen saturation values (e.g., the most recent 60 seconds of oxygen saturation values). In some embodiments, a set of rules may be applied to determine whether to suppress or switch off the lost respiration alarm. Although it will be understood that any suitable rules may be applied, in some embodiments, the rules may be based on overall increase in the average oxygen saturation value, a slope of the average oxygen saturation value, any other suitable metric or value, or any combination thereof.

If it is determined that a lost respiration alarm should be suppressed or switched off at step 706, processing may continue to step 704. At step 704, monitor 10 may set a lost respiration alarm status to post normally. Although a lost respiration alarm status may be set to post normally in any suitable manner, in an embodiment, any alarms (e.g., visual alarms, audio alarms, and alarm messages) relating to lost respiration may be reset. At the conclusion of step 704, processing may return to step 512 of FIG. 5.

If it is determined that a lost respiration alarm should not be suppressed or switched off at step 706, processing may continue to step 708. At step 708, monitor 10 may analyze oxygen saturation values to determine whether to set and/or adjust a lost respiration alarm. For example, if oxygen saturation is decreasing this may confirm that a patient has compromised respiration. In some embodiments, if the recently determined oxygen saturation value has decreased from the previous value by more than a threshold, a lost respiration alarm may be confirmed or adjusted. In some embodiments, a trend of recently determined oxygen saturation values may be analyzed. Although the trend of recently determined oxygen saturation values may be analyzed in any suitable manner, in an embodiment, an average oxygen saturation value (e.g., for 5 second segments of oxygen saturation) may be calculated for a snapshot of recently determined oxygen saturation values (e.g., the most recent 60 seconds of oxygen saturation values). In some embodiments, a set of rules may be applied to determine whether to confirm or adjust the lost respiration alarm. Although it will be understood that any suitable rules may be applied, in some embodiments, the rules may be based on overall decrease in the average oxygen saturation value, a slope of the average oxygen saturation value, any other suitable metric or value, or any combination thereof. In some embodiments, the elapsed time that the oxygen saturation falls below a threshold may be analyzed to determine whether the lost respiration indication should be confirmed or adjusted. For example, an analysis of the duration that the oxygen saturation is below a threshold, the degree to which the oxygen saturation is below a threshold, or both may be made. In this example, an integral may be calculated representative of the area between an oxygen saturation measurement over time and a corresponding threshold. When the integral exceeds a threshold, the respiration lost indication may be confirmed or adjusted.

It will be understood that steps 706 and 708 may be combined into a single step in which the oxygen saturation is analyzed to determine whether to determine that a respiration lost condition exists. For example, the trend of the oxygen saturation may be analyzed to determine if it is increasing. In this case, if the trend is increasing, monitoring system 10 may determine that the respiration is not lost and will suppress the respiration lost alarm. If the trend is not increasing, then monitoring system 10 may determine that respiration is lost and activate an appropriate alarm. In other words, two separate steps (i.e., 706 and 708) are not required to implement this functionality.

In some embodiments at step 706, 708, or both, the oxygen saturation data may be analyzed to determine if oxygen saturation has changed according to a dynamically calculated threshold. For example, in some embodiments, the threshold may be a function of the measured oxygen saturation, itself (e.g., at 100% initial saturation, the threshold may be set to a decrease of at least 6%, at 95% initial saturation, the threshold may be set to a decrease of at least 4%).

If it is determined that the lost respiration alarm should not be confirmed or adjusted at step 708, processing may continue to step 710. At step 710, monitor 10 may maintain the lost respiration alarm at its current state. Alternatively, to the extent the respiration lost alarm is currently in an active state, then at step 710, monitoring system 10 may change the state to inactive. At the conclusion of step 704, processing may return to step 512 of FIG. 5.

If it is determined that a lost respiration alarm should be confirmed or adjusted at step 708, processing may continue to step 712. At step 712, monitor 10 may confirm or adjust a lost respiration alarm. In some embodiments, a lost respiration alarm may be a binary (e.g., on/off) alarm, a graded (e.g., percentage alarm, severity alarm, etc.), any other suitable alarm, or any combination thereof. Although it will be understood that a lost respiration alarm may be confirmed or adjusted in any suitable manner, in some embodiments, if the lost respiration alarm was previously in an off state (e.g., off for a binary alarm or 0% for a percentage graded alarm) the alarm status may be turned on and/or set to an appropriate graded value. In some embodiments, if the lost respiration alarm was previously in an on state (e.g., on for a binary alarm or greater than 0% for a percentage graded alarm) the alarm status may be adjusted in the case of a graded alarm (e.g., by increasing or decreasing the percentage of a percentage graded alarm). Although a value for a graded alarm may be set in any suitable manner, in some embodiments, a graded alarm may be based on the respiration rate analysis of step 702 (e.g., the severity of the lost respiration condition), the oxygen saturation analysis of step 708 (e.g., the percentage drop in oxygen saturation), any other suitable parameter, or any combination thereof. At the conclusion of step 712, processing may return to step 512 of FIG. 5.

In some embodiments, if at step 702, it is determined that respiration is lost, and if at step 706, 708, or both, it is determined that the oxygen saturation does not indicate that respiration is actually lost, a respiration lost alarm may still be activated after, for example, a predetermined time delay. For example, if for some predetermined number of data buffers within a particular time period, monitoring system 10 determines at step 702 that a respiration lost condition exists based on an analysis of respiration information, then despite what is determined at step 706, 708, or both, a respiration lost alarm will be activated.

Referring again to FIG. 5, at step 512 monitor 10 may provide a result based on the determination of lost respiration alarm check at step 510. Although it will be understood that monitor 10 may provide a result in any suitable manner, in an embodiment, monitor 10 may provide a visual alarm, an audible alarm, a transmitted alarm, any other suitable alarm, or any combination thereof.

Although it will be understood that a visual alarm may be provided in any suitable manner, in some embodiments, a visual alarm may be provided on display 20 as an icon, text, intermittent flashing, changes to display color, any other suitable visual indication of an alarm, or any combination thereof. In the instance of a graded alarm, the icon, text, intermittent flashing, display color, or other indication may be adjusted based on the severity of the graded alarm condition.

Although it will be understood that an audible alarm may be provided in any suitable manner, in some embodiments, an audible alarm may be provided by speaker 22 as a spoken message, alarm sound, any other suitable audible indication of an alarm, or any combination thereof. In the instance of a graded alarm, the spoken message, alarm sound, or other indication may be adjusted based on the severity of the graded alarm condition.

Although it will be understood that a transmitted alarm message may be provided in any suitable manner, in some embodiments, a transmitted alarm message may be provided by data output 84 to any suitable receiving device such as a central nurse station, smart phone, computing unit, medical pager, medical database, any other suitable receiving device, or any combination thereof. In the instance of a graded alarm, the transmitted message may be adjusted based on the severity of the graded alarm condition.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed is:

1. A computer-implemented method comprising:
receiving a photoplethysmograph (PPG) signal;
generating, using processing circuitry, a respiration information signal based on the PPG signal;
generating, using the processing circuitry, oxygen saturation information based on the PPG signal; and
activating, using the processing circuitry, a respiration lost alarm based on the respiration information signal and on the oxygen saturation information.

2. The method of claim 1, wherein generating oxygen saturation information comprises identifying a decrease in oxygen saturation that exceeds a threshold.

3. The method of claim 2, wherein the threshold is based on an initial oxygen saturation value.

4. The method of claim 1, wherein generating oxygen saturation information comprises identifying a decrease in oxygen saturation that exceeds a threshold, the decrease occurring within a predetermined period of time.

5. The method of claim 1, wherein generating a respiration information signal comprises identifying a portion of the PPG signal that is not suitable for determining respiration information.

6. The method of claim 1, further comprising identifying in the respiration information signal a decrease in amplitude.

7. The method of claim 6, wherein the identifying in the respiration information signal a decrease in amplitude comprises identifying in the respiration information signal a decrease in amplitude for at least a predetermined period of time.

8. A system comprising:
an input for receiving a photoplethysmograph (PPG) signal; and
processing circuitry configured for:
generating a respiration information signal based on the PPG signal,
generating oxygen saturation information based on the PPG signal,
activating a respiration lost alarm based on the respiration information signal and on the oxygen saturation information.

9. The system of claim 8, wherein the processing equipment is further configured for identifying a decrease in oxygen saturation that exceeds a threshold.

10. The system of claim 9, wherein the threshold is based on an initial oxygen saturation value.

11. The system of claim 8, wherein the processing equipment is further configured for identifying a decrease in oxygen saturation that exceeds a threshold, the decrease occurring within a predetermined period of time.

12. The system of claim 8, wherein the processing equipment is further configured for identifying a portion of the PPG signal that is not suitable for determining respiration information.

13. The system of claim 8, wherein the processing equipment is further configured for identifying in the respiration information signal a decrease in amplitude.

14. The system of claim 13, wherein the wherein the processing equipment is further configured for identifying in the respiration information signal a decrease in amplitude for a predetermined period of time.

15. A non-transitory computer-readable medium having computer program instructions stored thereon for performing the method comprising:
receiving a photoplethysmograph (PPG) signal;
generating a respiration information signal based on the PPG signal;
generating oxygen saturation information based on the PPG signal; and
activating a respiration lost alarm based on the respiration information signal and on the oxygen saturation information.

16. The computer-readable medium of claim 15, wherein generating oxygen saturation information comprises identifying a decrease in oxygen saturation that exceeds a threshold.

17. The non-transitory computer-readable medium of claim 16, wherein the threshold is based on an initial oxygen saturation value.

18. The non-transitory computer-readable medium of claim 15, wherein generating oxygen saturation information comprises identifying a decrease in oxygen saturation that exceeds a threshold, the decrease occurring within a predetermined period of time.

19. The non-transitory computer-readable medium of claim 15, further comprising identifying in the respiration information signal a decrease in amplitude.

20. The non-transitory computer-readable medium of claim 19, further comprising identifying in the respiration signal the decrease in amplitude for at least a predetermined period of time.
